(19) United States
(12) Patent Application Publication
(10) Pub. No.: US 2011/0168177 A1
(43) Pub. Date: Jul. 14, 2011

(54) Anesthesia Simulator and Controller for Closed-Loop Anesthesia

(75) Inventor: Christopher W. Connor, Arlington, MA (US)

(73) Assignee: THE BRIGHAM AND WOMEN'S HOSPITAL, INC., Boston, MA (US)

(21) Appl. No.: 13/119,153

(22) PCT Filed: Sep. 13, 2009
(86) PCT No.: PCT/US09/56766

§ 371 Date: Mar. 15, 2011

Related U.S. Application Data
(60) Provisional application No. 61/097,790, filed on Sep. 17, 2008.

(51) Int. Cl. A61M 16/01

(52) U.S. Cl. 128/203.14

(57) ABSTRACT

Methods, systems, and software are disclosed for simulating and controlling anesthesia delivery and for implementing automatic closed-loop anesthesia with an inhaled anesthetic agent. The methods involve using a physiological simulation to forward calculate the effects of an inhaled anesthetic agent, and searching for gas administration characteristics, such as fresh gas flow rates and anesthetic concentrations or partial pressures, that will achieve a desired physiological anesthetic concentration or effect with minimal use of anesthetic agent and minimal fresh gas flow rate.

### CROSS-REFERENCE TO RELATED APPLICATIONS

This application claims priority to, and the benefit of, U.S. Provisional Patent Application No. 61/097,790, filed Sep. 17, 2008, the contents of which are incorporated by reference herein in their entirety.

### TECHNICAL FIELD

The invention relates generally to the field of anesthesia administration and, more particularly, to simulators and controllers for controlling anesthesia administration.

### BACKGROUND OF THE INVENTION

Many surgical procedures require that the patient be anesthetized, so that the patient is unable to feel sensation, either locally or generally. In many cases, general anesthesia is induced by inhalation of an anesthetic agent. Many common inhalational anesthetic agents are volatile short-chain halogenated hydrocarbons that are liquid at room temperature but are easily vaporized and carried by a gas stream. Gaseous inhaled anesthetic agents may also be used; two of the more common gaseous agents include nitrous oxide and xenon.

Typically, an anesthesia machine creates a mixture of fresh gas (pure oxygen or a mixture of oxygen, air, and other gases) and an anesthetic agent. That mixture flows into an anesthesia circuit—the collection of tubing, machinery, and associated hardware that delivers the gas mixture to the patient's lungs for inhalation. With each breath, the patient absorbs some, but not all, of the gas in the anesthesia circuit. When the patient exhales, waste gases like carbon dioxide are removed by a chemical scrubber, and the remainder of the gas is recirculated to the patient.

The anesthetic agent enters the body by gas exchange in the alveoli of the lungs. From the alveoli, the agent is carried by the arterial blood flow to the rest of the body. The arterial blood flow exposes substantially the entirety of the patient's body to the anesthetic agent, but the majority of the anesthetic effect produced by the agent depends on its concentration within certain organs, like the brain. For purposes of description, calculation, and modeling, organs and systems of the body that absorb anesthetic agent similarly are often grouped into conceptual “compartments.” The most common compartments are the alveoli, arterial blood, venous blood, muscle, fat, and the vessel-rich group (VRG). Of those groups, the VRG is of particular importance. Specifically, the vessel-rich group represents the organs of the body that are highly perfused with blood, like the liver, kidneys, and the brain, and the nature and degree of the anesthetic effect produced by any agent is most closely correlated to its concentration within the VRG.

However, when administering anesthesia to a patient, the anesthesiologist cannot manipulate the concentration of anesthetic agent within the VRG (or any other compartment) directly. Instead, the anesthesiologist can only alter the fresh gas flow (FGF), the percentage or partial pressure of anesthetic agent in the anesthesia circuit (i.e., the vaporizer setting), and the mode of ventilation. In practice, most anesthesiologists will initially set a relatively high FGF of several liters per minute, will set an anesthetic agent concentration in the circuit that is equal to the desired steady-state concentration in the VRG, and will simply wait for the anesthetic concentration in the VRG to reach the desired steady-state concentration. (The anesthetic concentration in the VRG is usually determined indirectly by measuring the end tidal concentration of anesthetic agent, i.e., the concentration of anesthetic agent in exhaled breath.)

While setting the steady-state desired anesthetic agent concentration with a high fresh gas flow and waiting for the VRG concentration to equilibrate with the concentration of anesthetic agent in the circuit is a simple and easily implemented technique, it is also wasteful. Anesthetic agent is expensive, and with that technique, much of it passes through the circuit without having any effect on the patient. The problem is particularly acute when the anesthetic agent is xenon, a rare and expensive elemental gas that requires a concentration in the VRG of approximately 70% for full anesthetic effect.

There is an alternative form of anesthetic administration called closed-loop anesthesia. In closed-loop anesthesia, the patient is given just enough FGF to satisfy the metabolic oxygen demand and just enough anesthetic agent to achieve the desired VRG concentration, and only the gases that are actually taken up by the patient are replaced in the anesthesia circuit. Closed-loop anesthesia is very efficient, and saves material and cost, but is difficult to administer properly. In part, this is because the flow rates and concentrations used in closed-loop anesthesia are very different than those used with the standard techniques, and can be difficult for many anesthesiologists to comprehend and implement. Therefore, despite cost and material savings, closed-loop anesthesia is not widely used.

### SUMMARY OF THE INVENTION

One aspect of the invention relates to a method for controlling the administration of an inhaled anesthetic agent to meet a desired physiological target or effect. The method comprises determining one or more gas administration characteristics, including at least a concentration or partial pressure of the inhaled anesthetic agent, using a physiological simulator that simulates the uptake and behavior of the inhaled anesthetic agent. In some embodiments, the gas administration characteristics may comprise a fresh gas flow rate and an anesthetic concentration or partial pressure, and the method may implement closed-loop anesthesia by determining the minimum fresh gas flow rate and anesthetic concentration that will meet the desired physiological target or effect.

Other aspects of the invention relate to anesthesia machines with controllers that perform the method described above, and to sets of machine-readable instructions on machine-readable media that are interoperable with machines to perform the method described above.

Additional aspects, features, and advantages of the invention will be described below.

### BRIEF DESCRIPTION OF THE DRAWINGS

The invention will be described with respect to the following drawing figures, in which like numerals represent like elements throughout the figures, and in which:

FIG. 1 is a schematic view of an anesthesia machine with a controller according to one embodiment of the invention;

FIG. 2 is an illustration of a user interface for the controller of FIG. 1;

FIG. 3 is a flow diagram illustrating the tasks performed by the controller of FIG. 1; and

FIG. 4 is a flow diagram illustrating a method for target-based control over anesthetic agent concentration in the VRG.

### DETAILED DESCRIPTION

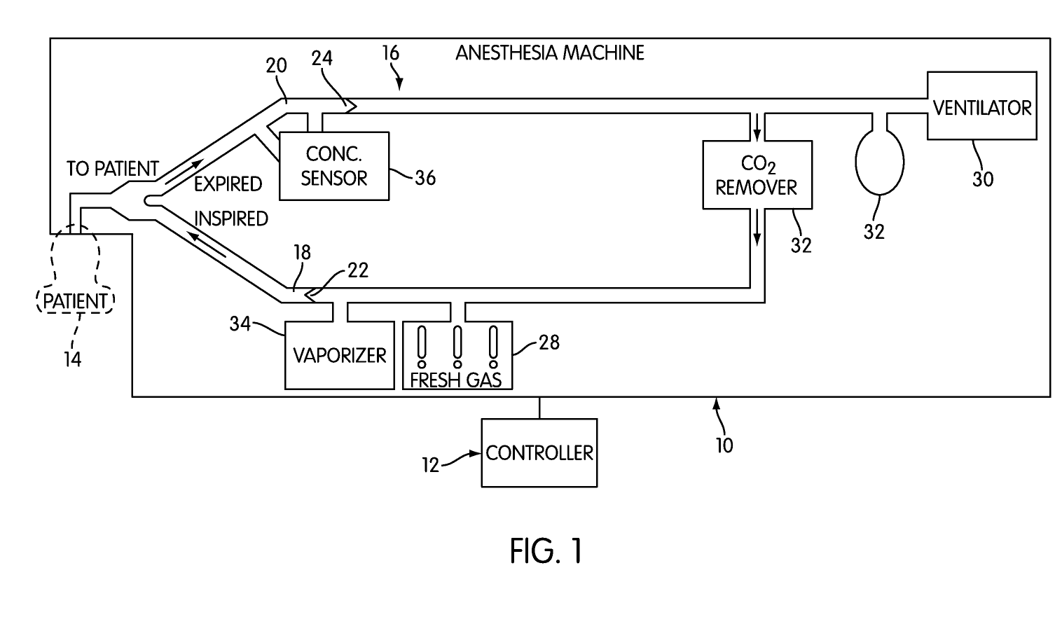

FIG. 1 is a schematic view of an anesthesia machine, generally indicated at 10, with an anesthetic controller 12. The anesthesia machine 10 provides a precisely controlled mixture of fresh gas (e.g., oxygen and/or air) and gaseous or volatile anesthetic agents to a patient 14. The controller 12 determines gas administration characteristics (such as fresh gas flow and volatile anesthetic agent percentage or partial pressure) that are likely to result in a desired physiological concentration of the anesthetic agent, or a desired physiological effect, by simulating the uptake and behavior of an inhaled anesthetic agent in a particular gas flow.

In the description that follows, it should be understood that the anesthesia machine 10 and anesthetic controller 12 may be used in a variety of settings, including operating rooms or theaters, intensive care units, and any other setting in which such devices are conventionally used. Additionally, although this disclosure refers to “anesthesia” for convenience and simplicity in description, anesthesia machines 10 and controllers 12 according to embodiments of the invention may be used for a variety of purposes, and need not be limited to producing an anesthetized state for surgical purposes. For example, an anesthesia machine 10 and controller 12 may be used to sedate patients in an intensive care unit. Moreover, although this disclosure focuses on the simulation and control of inhaled anesthetic agents, methods and systems according to embodiments of the invention may be used to simulate and control the effects of any inhaled agent with similar pharmacokinetics.

The anesthesia machine 10 illustrated in FIG. 1 is a schematic and generalized anesthesia machine. Many different types of anesthesia machines are known in the art, and essentially any type of anesthesia machine may be used in embodiments of the invention. The anesthesia machine 10 includes an anesthesia circuit, which is generally indicated at 16 in FIG. 1. The anesthesia circuit 16 contains an inspiratory side 18, in which gas is directed from the anesthesia machine 10 into the patient 14, and an expiratory side 20, in which gas exhaled by the patient 14 is received in the circuit 16. Each side of the circuit 18, 20 includes a one-way valve 22, 24. The one-way valve 22 on the inspired side 18 prevents gas from flowing opposite the inspiratory direction; the one-way valve 24 on the expired side prevents gas from flowing opposite the expiratory direction (both directions are indicated by arrows in FIG. 1).

The two sides 18, 20 of the circuit 16 connect to a device such as an endotracheal tube (not shown in FIG. 1), which brings the flow to the patient's lungs. Alternatively, the circuit 16 may deliver gas through a mask or another breathing device. On the other end of the circuit 16, a carbon dioxide remover 26 connects the inspiratory and expiratory sides 18, 20 such that carbon dioxide-containing expired gas is stripped of its carbon dioxide before being recycled into the inspired side 18 of the circuit 16. In a typical embodiment, the carbon dioxide remover 26 may be a canister of soda lime (a mixture of metal hydroxides, including calcium hydroxide, sodium hydroxide, and potassium hydroxide) and its associated hardware.

On the inspiratory side 18 of the circuit 16, the anesthesia machine 18 provides a fresh gas source 28. A fresh gas source 28 generally takes gases (e.g., oxygen, air, and nitrous oxide) at higher pressure from their respective sources and allows the anesthesiologist to control the mixture and flow rates of fresh gases that are introduced into the circuit 16 and, consequently, the patient 14. The sources of fresh gas may be either large, remote sources of compressed gases, or individual bottles of the respective gases that are attached directly to the anesthesia machine 10. Depending on the embodiment, the fresh gas source 28 may be manually controlled, controlled automatically by the controller 12, or may be configured for either manual or automatic control depending on the situation and operating mode. A mechanical ventilator 30 and reservoir bag 32 may also be connected to the circuit 16 for automatic and manual ventilation of the patient 14, respectively.

Also connected to the circuit 16 on the inspiratory side 18 is a vaporizer 34 that introduces desired amounts of a volatile anesthetic agent into the circuit 16 for delivery to the patient 14. Many different volatile anesthetic agents are known in the art, and any of them may be used in embodiments of the invention. Examples of volatile agents include ether, halothane, enflurane, isoflurane, sevoflurane, and desflurane. As was noted above, these agents are generally liquid at room temperature, although they have high vapor pressures and can readily be caused to vaporize. Several types of vaporizers are known in the art, and any may be used in embodiments of the invention.

In one common type of vaporizer, called an overflow bypass vaporizer, some of the fresh gas flow goes straight through the vaporizer while a small amount of the gas flow is diverted into a chamber filled with vaporized anesthetic agent and is allowed to become saturated with the anesthetic agent before rejoining the main gas flow. The percentage or partial pressure of anesthetic agent in the gas flow is altered by altering how much of the gas flow is diverted into the vaporization chamber. The vaporizer generally maintains constant temperature and pressure conditions within the vaporization chamber, so that a known quantity of anesthetic agent is in the vaporization chamber at all times.

In a second type of vaporizer, the amount of gas flowing past the vaporizer is sensed and liquid anesthetic agent is injected directly into the gas flow in such a way that it will vaporize. The injectors for this second type of vaporizer are typically electrically controlled, making this type of vaporizer particularly compatible with automatic control.

Regardless of its mode of operation, each type of vaporizer generally provides the anesthesiologist with a standard set of controls, typically including a knob or dial that, when actuated, changes the percentage or partial pressure of the anesthetic agent in the gas flow. In some portions of this description, the term “vaporizer setting” may be used to mean percentage or partial pressure of anesthetic agent in the gas flow.

Because the vapor pressure and behavior of each volatile anesthetic agent are different, a different vaporizer is usually provided for each agent, and an anesthesia machine 10 may include several vaporizers, each one of them different and designed for a particular volatile anesthetic agent. While vaporizers for most of the anesthetic agents are mechanically similar, vaporizers for some anesthetic agents may be different, even if their controls and outward appearance are similar. For example, desflurane has a much higher vapor pressure than the other volatile agents; therefore, most desflurane vaporizers are electrically heated and pressurized, typically holding the liquid at a pressure of about two atmospheres. The liquid is allowed to boil off as required.

The anesthesia machine 10 may also include various sensors, such as sensor 36, that indicate the end-tidal concentration of the various gases in the expired side 20 of the anesthesia circuit 16. The sensor 36 of FIG. 1 is illustrated as returning its sampled gas to the circuit, which is advantageous in closed-loop anesthesia, because exhausting sensor-sampled gas can waste on the order of a few hundred milliliters of gas per minute. However, in some embodiments, the sampled gas flowing into the sensor 36 may not be returned to the circuit 16. Additionally, any number of physiological sensors may be connected or coupled directly or indirectly to the anesthesia machine 10 to measure any relevant respiratory, cardiac, or general physiological parameters or signs. A non-exclusive list of physiological sensors that may be used include an EKG, pulse oximeter, blood pressure sensor (either a non-invasive cuff or an invasive arterial line), in-circuit oxygen sensor, and end-tidal capnometer Infrared spectrometry of inhaled and exhaled gases may also be used. As those of skill in the art will understand, the anesthesia machine 100 may include other sensors as necessary to monitor its own performance.

The controller 12 may directly control the anesthesia machine 10, or it may act as a non-controlling simulator to provide advisory information to the anesthesiologist. In particularly advantageous embodiments, the controller 12 may have two modes of operation: (1) a standard mode of operation, in which the anesthesiologist can control the fresh gas flow and anesthetic agent concentration as usual using the controller; and (2) a target-based control mode, in which the anesthesiologist sets a target anesthetic concentration in a physiological compartment, or some other physiological target, and the controller 12 controls the fresh gas flow and anesthetic agent concentration according to an algorithm to meet that target. For example, in target-based control mode, the controller 12 may automatically control the fresh gas flow and anesthetic agent concentration to meet a desired target VRG anesthetic concentration. In either mode, the controller 12 may be in direct electronic control of the anesthesia machine 10, or it may not directly control the anesthesia machine, instead acting as a simulator and advising the anesthesiologist of the appropriate settings in any given moment.

Generally speaking, when in target-based control mode, the controller 12 controls the fresh gas flow and anesthetic agent concentrations by using a physiological simulator to “forward calculate” the physiological effects of various sets of fresh gas flows and agent concentrations and searching for sets of parameters that will bring the desired physiological concentration to the desired level in the shortest period of time. Essentially, when in target-based control mode, the controller 12 provides automatic closed-loop anesthesia, searching for the lowest fresh gas flow that will meet basic metabolic oxygen demand and achieve the desired anesthetic concentrations, and replacing in the anesthetic circuit 16 only those gases that are taken up by the patient 14. In some embodiments, the controller 12 may perform other functions as well, including calculating the total cost of the anesthetic agent consumed, and calculating the time that it will take to bring a patient from an anesthetized state to the point of emergence from anesthesia.

The controller 12 may be implemented entirely in hardware with physical controls, it may be implemented entirely in software with virtual controls that are operable through a graphical user interface (GUI), or it may be implemented in some combination of hardware and software. Additionally, although shown as a separate element in FIG. 1, the functions of the controller may be integrated into the anesthesia machine 10 and may use its monitors and displays.

If implemented in software, the controller 12 may be in the form of machine-readable instructions on a machine-readable medium that are interoperable with a machine to perform the functions of the controller 12. Many different types of machines may implement these sorts of instructions, including general-purpose desktop and laptop computers, application specific integrated circuits (ASICs), and other special purpose computers. In general, when the term “computer” is used in this description, it should be construed to refer to any machine with the capability to implement the described functions. Machine-readable instructions may be written in any programming language and compiled into any form. Examples of programming languages that may be used to implement the controller 12 include compiled languages like C, C++, Java, J++, and assembly code, and interpreted languages, such as Python. Machine-readable media include magnetic media (e.g., hard disk drives, floppy disk drives), optical media (e.g., CD, CD-R, DVD), and solid state memory (e.g., flash memory, solid state drives, random access memory, read-only memory, etc.). The term “machine-readable media” should also be construed to include media such as so-called “golden master” disks, which are intended to be used to install software on a plurality of machines, and may or may not contain machine-readable instructions that are directly executable by a machine.

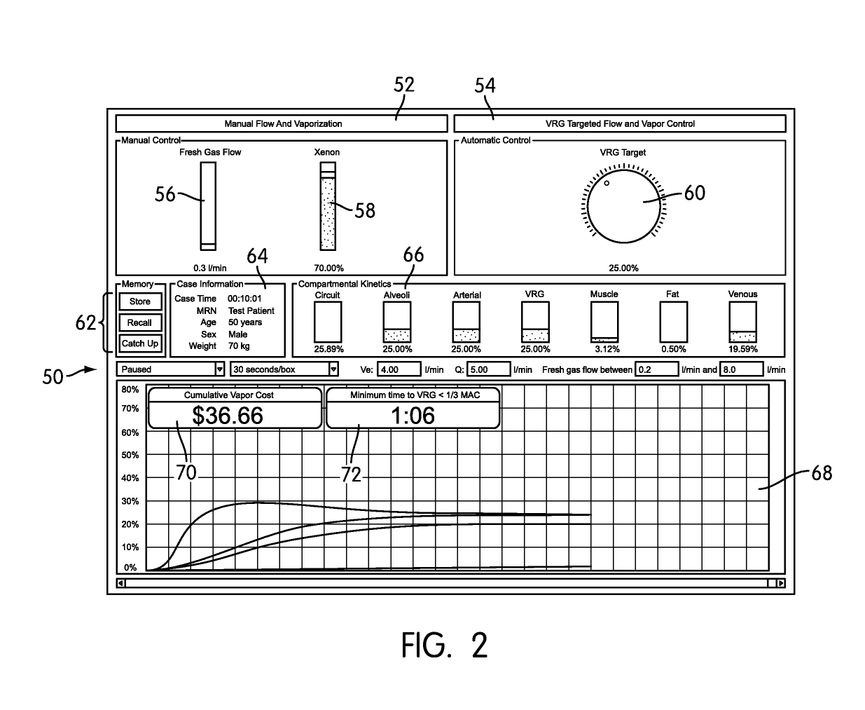

FIG. 2 is an illustration of a one embodiment of a graphical user interface, generally indicated at 50, for the controller 12. Near the top of the interface, mode selector buttons 52, 54 are provided that allow the user to select whether to use manual mode (selector button 52) or whether to use target-based control mode (selector button 54). In manual mode, the user uses the FGF slider 56 and the anesthetic agent slider 58 to control the fresh gas flow and the anesthetic agent concentration directly. In target-based control mode, the user uses the target knob 60 on the right side of the interface to set target VRG anesthetic agent concentration, and the FGF and anesthetic agent sliders 56, 58 and the corresponding gas administration characteristics are controlled automatically to reach the selected target.

Below the mode selectors 52, 54 and flow and target controls 56, 58, 60, the interface 50 displays the current status. On the left are a set of memory controls 62 that are useful particularly in simulation for saving and recalling particular states. To the right of that, a case information display area 64 displays particular case information such as the case elapsed time, the patient's medical record number, and the patient's age, weight, and sex. To the right of the case information display area 64, is a compartmental kinetics display area 66 that displays the anesthetic agent concentration in each relevant compartment: in the anesthetic circuit, the alveoli, the arterial blood, the VRG, muscle, fat, and the venous blood.

Below the compartmental kinetics display area 66 is a graph 68 that shows the history of the compartmental kinetics in graphical form. Just above the graph 68, the user can select the timing factor, which can be used in simulations to speed up or slow down the simulation; the visual characteristics of the graph, for example, the amount of elapsed time per box or gradation; and certain physiological and ventilation parameters, such as the minute ventilation (Vₑ in liters per minute of gas), the patient's cardiac output (Q, in liters per minute of blood), and the upper and lower bounds of possible fresh gas flow rates. In the illustration of FIG. 2, the controller 12 is running in target-based control mode to administer xenon with a target VRG concentration of 25%. In the illustrated instant, the fresh gas flow is set by the controller at 0.3 L/min, the xenon concentration is set at 70%, and the compartmental kinetics show that xenon has reached the desired steady state concentration of 25% in the VRG. Furthermore, the patient's Vₑ is 4 L/min and his cardiac output is 5 L/min. Fresh gas flows between 0.2 and 8 L/min are permitted in the illustration of FIG. 2. It should be understood that even when the controller 12 is in target-based control mode, it may be configured to move the FGF slider 66 and anesthetic agent slider 68 automatically to indicate what the present settings are.

Inset into the graph are two indicators. The first indicator, a cost indicator 70, indicates the total cost of the anesthetic agent used. The second indicator 72 gives an estimate of the amount of time it would take to bring the patient out of anesthesia if delivery was stopped at that moment and only fresh gas was delivered. Specifically, each anesthetic agent has a particular mean anesthetic concentration (MAC) value in the VRG at steady state at which 50% of a population did not move in response to a standard stimulus (e.g., a midline laparotomy). The MAC value for an anesthetic agent is used as an indicator of what the target VRG concentration should be for a particular anesthetic agent in most patients. One-third of the MAC value is considered to be the VRG concentration at which most patients are minimally anesthetized. Therefore, the amount of time it would take a patient to reach one-third of the MAC value is used as an estimate of the amount of time it would take for the patient to regain consciousness after anesthesia. In the illustration of FIG. 2, the second indicator 72 estimates that it would be one minute and six seconds for the patient to arrive at one-third of the MAC value of xenon delivery was stopped.

The interface 50 may have essentially any look or any set of controls, and certain functions shown as being performed by multiple controls may be consolidated into a single control. Moreover, the look of the controls may be different from operating system to operating system. However, although any look or controls may be used, it may be helpful in some embodiments to mimic the look or function of the types of controls that are found on typical anesthesia machines.

As was noted above, methods and systems according to embodiments of the invention typically seek to maintain a particular VRG anesthetic concentration with minimal usage of gas by creating a physiological model and searching for appropriate gas administration characteristics, such as fresh gas flow rates and circuit anesthetic concentrations, that achieve and maintain the target VRG concentration with minimal usage of gas.

In order to perform the simulation, embodiments of the invention employ a state model to determine and predict the uptake and distribution of anesthetic agents in the body. Suitable state models include those articulated by Eger (e.g., Eger, E. I., “Respiratory and Circulatory Factors in Uptake and Distribution of Volatile Anaesthetic Agents,” Brit. J. Anaesth. (1964), 36, p. 155.) and Mapleson (e.g., Mapleson, W. W., “Mathematical Aspects of the Uptake, Distribution, and Elimination of Inhaled Gases and Vapours,” Brit. J. Anaesth. (1964), 36, p. 129.). Those articles are incorporated by reference herein in their entireties, and particular discrete numerical implementations of those models will be described in more detail below.

Using a state model, the controller 12 determines what the concentration of anesthetic agent in the compartments of the body will be for a particular FGF rate and vaporizer setting. In principle, one could simply run the state model for every possible fresh gas flow and vaporizer setting at intervals of, for example, one second to achieve target-based control. However, doing so is extremely computationally intensive.

In order to simplify the computational problem of simulating the effect of fresh gas flow rate and vaporizer setting on VRG anesthetic concentration, in embodiments of the invention, at least one of those two gas administration characteristics is represented in such a way that its effects scale linearly, and thus, the effect of changing that characteristic can be determined easily.

Specifically, in embodiments of the invention, transfer functions are constructed that linearize and simplify the process of determining an appropriate anesthetic agent concentration or partial pressure given a particular fresh gas flow. These transfer functions are unit distribution functions (UDFs) that describe the anesthetic agent concentration in the VRG if a defined “unit” concentration of anesthetic agent is introduced into the circuit for a defined “unit” of time starting at time zero with a given fresh gas flow rate. After the defined unit of time, the fresh gas flow rate is set to the minimum allowed fresh gas flow rate. For example, a UDF may describe the VRG concentration if a 1% vaporizer setting is used for one second. A single UDF is a nonlinear curve—it peaks and then declines. However, the relationship between UDF curves in a family of UDFs, i.e., UDFs that describe different vaporizer settings with the same fresh gas flow rate, is linear, and so a UDF can be linearly scaled.

For example, the response to a 2% concentration of anesthetic agent in the circuit at a given fresh gas flow rate can be determined essentially by multiplying the 1% UDF at that fresh gas flow rate by two. UDFs can be precalculated for every allowed fresh gas flow rate, thus greatly simplifying the computational tasks. Moreover, in some aspects of the calculations, only the peak concentration and the time at which the peak occurs may be relevant, thus further simplifying computational and data storage requirements. Particular methods for creating and using UDFs will be described below in more detail.

As was described briefly above, in embodiments of the invention, the fresh gas flow is typically defined in a discrete form, such that it can have a number of discrete settings between a defined maximum and a defined minimum. Because of the nature of the physiological modeling, defining the fresh gas flow in this way is computationally efficient. Specifically, in closed-loop anesthesia, it is reasonable to assume that in most cases, the fresh gas flow will either be at the defined minimum value or the defined maximum value. If neither one of those settings is appropriate, then a search can be made of the other possible fresh gas flow settings to find the minimum flow setting that will achieve the desired target.

In some embodiments, the fresh gas flow could be treated as a continuous variable and fresh gas flow values that will achieve a desired target VRG concentration could be solved for using a piecewise linear method, like the Newton-Raphson method. However, because of the assumptions that can be made about fresh gas flows in closed-loop anesthesia, it will generally be more efficient simply to establish a number of discrete fresh gas flow settings and to choose fresh gas flow settings as described above.

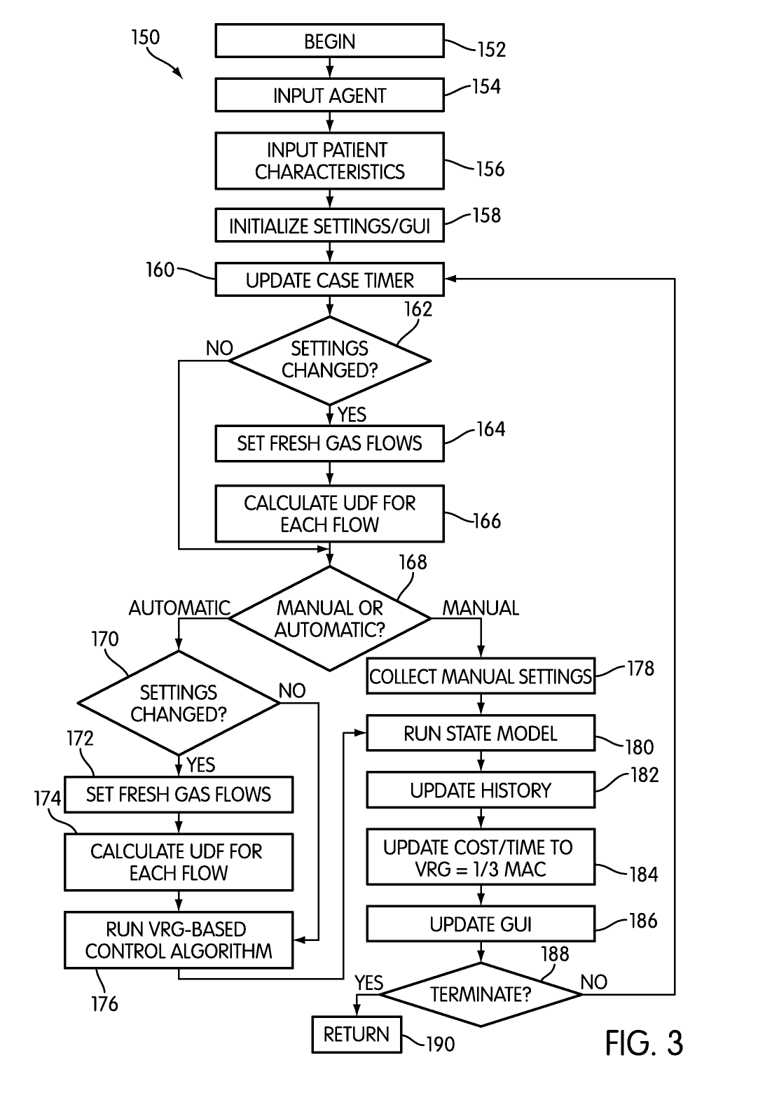

These concepts will be described in more detail with respect to FIG. 3, a schematic flow diagram of a method, generally indicated at 150, of determining gas administration characteristics for a volatile or gaseous anesthetic agent by physiological simulation, the basic method implemented by the controller 12. Method 150 begins at task 152 and continues with task 154.

Typically, the process of anesthetizing any particular patient would begin with the anesthesiologist selecting an anesthetic agent and inputting certain information about the patient, including, for example, the patient's age, weight, medical record number, and any other clinically relevant information. In some embodiments, the controller 12 may be connected via a network to a database that contains patient information, and the anesthesiologist may only need to enter a patient identification number or medical record number to initiate a process whereby other necessary information is retrieved directly from the database by the controller 12.

In task 154, the anesthesiologist inputs the name or identifier of the anesthetic agent that is to be used. This may be done, for example, by allowing the anesthesiologist to choose the name of the agent from a list or selection box. The choice of anesthetic agent would generally be left to the anesthesiologist, and would typically be a matter of medical judgment considering the patient, the procedure that is to be performed, and other conventional factors.

It should be understood that although this description focuses on the use of one anesthetic agent per procedure, there are clinical situations in which an anesthesiologist may switch a patient from one anesthetic agent to another during a procedure, and embodiments of the invention may accommodate the use of multiple agents used in succession. Some examples of situations in which multiple successive agents may be used include: (1) patients experiencing bronchial constriction, who may be switched to sevoflurane, because of its bronchial-dilating effects; (2) situations in which the anesthesiologist may use a less expensive anesthetic for most of a long procedure, and then may switch to an agent such as desflurane, hoping for a faster emergence from anesthesia; (3) pediatric patients, who may be induced by mask inhalation of sevoflurane before being switched to another anesthetic during the procedure to avoid certain pediatric sevoflurane complications that may occur when the patient emerges from anesthesia; and (4) any other circumstance in which the anesthesiologist believes that the patient would be better managed with another agent. If the agent is switched during the procedure, a second instance of method 150, or a relevant portion of method 150, could be executed in parallel with the first instance of method 150. This second instance of method 150 would track and display the gradual dissipation of the first anesthetic agent, while the new agent would be under active control. The effect of each agent would then be calculated relative to its MAC value. Generally speaking, once normalized for the different MAC values of the different agents, the effects of two anesthetic agents can be considered to be additive. Once an anesthetic agent has been selected, method 150 continues with task 156.

In task 156, the anesthesiologist inputs any patient characteristics that may be necessary, including the patient's weight, medical record number, and any other necessary or desirable information. Method 150 continues with task 158.

In task 158, the basic settings and the GUI are initialized. This task generally involves a number of sub-tasks, at least some of which may be specific to the operating system or platform on which method 150 is run. GUI initialization tasks may include instantiating the various graphical controls that form the interface 50 and associating those controls with the values of the various variables. Other general initialization tasks include setting the basic time rate of the system. Typically, the major tasks of a method like method 150 would be executed repeatedly at some defined interval, such as one second, so as to continuously provide correct vaporizer settings and fresh gas flow rates. However, as was described briefly above, when being used as a simulator, the time rate may be selected so as to speed up or slow down the simulation. In that case, the basic rate of time is generally scaled.

Additionally, task 158 includes the sub-tasks of setting the appropriate range of fresh gas flow values based on the defined minimum and maximum FGF settings, setting values for the patient, and setting values for the anesthetic agent. The basic set of values used in method 150 may be stored in a single record or struct defined, for example, as follows:

| | |
|---|---|
| typedef struct tag_SimData { | |
| TCHAR name[32]; | //Anesthetic agent name |
| TCHAR shortname[8]; | //Anesthetic agent short name |
| double MAC; | //Agent MAC value |
| double maxfVAP; | //Maximum vaporizer setting for the |
| | agent |
| double volatility; | //Volatility |
| double bottleCost; | //Agent bottle cost |
| double bottleSize; | //Agent bottle size |
| double lambda_blood; | //Agent solubility in blood |
| double lambda_vrg; | //Agent solubility in VRG |
| double lambda_mus; | //Agent solubility in muscle |
| double lambda_fat; | //Agent solubility in fat |
| double b_vrg; | //VRG partition coefficient |
| double b_mus; | //Muscle partition coefficient |
| double b_fat; | //Fat partition coefficient |
| double vol_ckt; | //Volume of the anesthetic circuit |
| double vol_alv; | //Volume of the alveoli |
| double vol_art; | //Volume of the arterial compartment |
| double vol_vrg; | //Volume of the VRG |
| double vol_fat; | //Volume of the fat |
| double vol_mus; | //Volume of the muscle |
| double vol_ven; | //Volume of the venous compartment |
| double Q; | //Patient cardiac output |
| double Ve; | //Patient minute ventilation |
| double dt; | //Basic time step interval |
| double minVfgf; | //Minimum fresh gas flow |
| double maxVfgf; | //Maximum fresh gas flow |
| int unitDuration; | //Duration of one unit of time |
| COLORREF agentColor; | //Agent color for graphing use |
| } SIMDATA; | |

The values for the anesthetic are generally known and may be set by taking the input from task 154, which establishes the agent that is being used, and setting the appropriate constants using, for example, a case statement. An example of the settings for sevoflurane are given in the code snippet below, which initializes the values of the struct pSim (of type SIMDATA, as defined above):

case 0: // Sevoflurane

strcpy(pSim->name,“Sevoflurane”);
strcpy(pSim->shortname,“SEV”);
pSim->MAC = 2.1;
pSim->maxfVAP = 8;
pSim->volatility = 183;
pSim->bottleCost = 180;
pSim->bottleSize = 250;
pSim->lambda_blood = 0.65;
pSim->lambda_vrg = 1.1;
pSim->lambda_mus = 2.4;
pSim->lambda_fat = 34;
pSim->agentColor = RGB(255,255,0);
// Sevoflurane graphing color is yellow.
setOK = 1;
break;

The patient-specific values may, for example, be defined as follows. In the following code, the variable identifier “mass” is the patient's mass in kilograms:

```
void patient(SIMDATA *pSim, double mass) {
    // Distribution of blood flow through VRG/MUS/FAT
    pSim->b_vrg = 0.76;
    pSim->b_mus = 0.18;
    pSim->b_fat = 0.06;
    // Volumes of compartments
    pSim->vol_ckt = 8.0;
    pSim->vol_alv = 2.5 * (mass/70);
    pSim->vol_art = 1.0 * (mass/70);
    pSim->vol_vrg = 6.0 * (mass/70);
    pSim->vol_fat = 14.5 * (mass/70);
    pSim->vol_mus = 33.0 * (mass/70);
    pSim->vol_ven = 1.0 * (mass/70);
    // Predict Q and Ve
    pSim->Q = (0.5 * pow(mass,0.75))/60;
    pSim->Ve = (0.16 * pow(mass,0.75))/60;
}
```

It should be understood that although certain elements, such as the compartment sizes, are represented as constants in the code above, in some embodiments, these values may be modified to suit the characteristics of the patient. This will be described below in more detail.

As was described above, when the controller 12 is used in target-based control mode, UDFs are calculated for each of the possible discrete fresh gas flow rates. Therefore, if the range of permissible fresh gas flow rates changes, or if any of the other settings relevant to the operation of target-based control mode change, some global mechanism for indicating that the UDFs and other simulation parameters should be recalculated is helpful. For that reason, a Boolean called is ValidAI is used to indicate whether or not the UDFs and other simulation parameters should be recalculated. Moreover, before any significant simulation tasks occur, the value of the is ValidAI flag is checked, and if the value is false, the necessary calculations or recalculations are performed. One of the last sub-tasks of task 158 would generally be to set the value of is ValidAI to false. Method 150 continues with task 160, in which the case timer is incremented.

Task 160 represents the beginning of the major loop of method 150, which is executed repeatedly until the termination of the method. The first task in that loop, shown as task 162 in FIG. 3, is to determine whether simulation-relevant settings have changed, requiring the recalculation of UDFs and re-setting of fresh gas flow rates. That determination is made by checking the state of the is ValidAI Boolean flag described above. In general, changes to the minimum and maximum fresh gas flow rates and changes to Q and Vₑ would cause a change in the value of is ValidAI, resulting in recalculation.

If the is ValidAI flag is set to false, the settings have changed, and it is necessary to re-set the fresh gas flow rates and recalculate the UDFs. Therefore, method 150 continues with tasks 164 and 166, the range of permissible fresh gas flows is set according to the newly-entered settings, and a UDF is calculated for every permissible fresh gas flow rate.

To set the range of permissible discrete fresh gas flow rates, for example, an array is created with the minimum fresh gas flow rate, the maximum fresh gas flow rate, and a number of intermediate fresh gas flow rates that are different from one another by some defined increment. The number of positions in the array is defined by taking the difference between the minimum and maximum values and dividing by the increment. An array might contain the minimum fresh gas flow rate in its first position, the maximum fresh gas flow rate in its last position, and the range of permissible fresh gas flow rates might differ by 0.1 liters per minute. Thus, for example, if the minimum fresh gas flow rate is 0.2 l/min and the maximum is 15.0 l/min, the array might contain [0.2, 0.3, 0.4, 0.5, 0.6, 0.7, 0.8, 0.9, 1.0, 1.1 . . . 15.0]. The increment between successive permissible fresh gas flows may be chosen such that each change in the fresh gas flow rate will produce a significant clinical effect, but the increment should be small enough to provide a great degree of flexibility and adjustability and to simulate the continuous adjustability of fresh gas flow found in most anesthesia machines.

As a first step in calculating a UDF for each permissible fresh gas flow, a long-duration UDF is first calculated and the peak of that UDF is located. Specifically, an arbitrary unit duration (for example, 3600 seconds) is set that will almost certainly be longer than the time it takes the VRG concentration to peak after a unit of anesthetic agent is administered. Once that unit time is set, a function is called to make a single UDF for the minimum permissible fresh gas flow over the time defined by the arbitrary unit duration. The UDF created by that function call is not stored; however, the function returns the time at which the VRG concentration peaks in that long-duration UDF, and the returned peak time is stored. Once the peak time is established, a loop is used to create a UDF for each fresh gas flow. However, when the rest of the UDFs are calculated, the unit duration for those UDFs is set equal to the time at which the first UDF peaked. This is a way of economizing on calculations: it is assumed that all other UDFs will peak higher and sooner than the initial long-duration UDF, so there is no need to calculate a UDF past the time that the long-duration UDF peaked. The peak time is also be used to determine how much memory to allocate for the array that holds the actual UDF values. The overall function that handles tasks 164 and 166 may be coded as follows:

```
void CpvDoc::updateAI( ) {
    int i;
    freeAI( );  //Release all memory and clear the previous UDFs
    /* Make the fresh gas flows */
    nvfgf = makeVfgf(NULL, &sim);
    vfgfRange = (double *)malloc(nvfgf*sizeof(double));
    if (vfgfRange == NULL) {
        AfxMessageBox(“VRG Allocation Failure”,
        MB_OK|MB_ICONSTOP);
        exit(EXIT_FAILURE);
    }
    makeVfgf(vfgfRange, &sim);
    /* Make the UDFs */
    udfLength = 3600;  //Set arbitrary long unit duration (1 hour)
    /* Make a long-duration UDF just to establish peak time/UDF
    length */
    udfLength = makeSingleUDF(NULL,udfLength,&sim,vfgfRange[0]);
    /* Allocate memory for the UDFs based on the peak time */
    udf = (double *)malloc(nvfgf * udfLength * sizeof(double));
    if (udf == NULL) {
        AfxMessageBox(“UDF Allocation Failure”,
        MB_OK|MB_ICONSTOP);
        exit(EXIT_FAILURE);
    }
    /* If memory allocated successfully, make the rest of the UDFs in a
    loop */
        for (i=0;i<nvfgf;i++)
        makeSingleUDF(udf+(i*udfLength),udfLength,&sim,
        vfgfRange[i]);
    /* Finally, set the isValidAI flag back to true */
    isValidAI = true;
}
```

As the code above indicates, in the present example, a function called makeSingleUDF is called to construct a single UDF. The function takes as its parameters a pointer to a storage location for the UDF data, the UDF length, the structure containing all of the simulation data, and the fresh gas flow rate for which the UDF is to be constructed. Each individual UDF is constructed by using a state model, as described above. One implementation of a state model is as follows:

```
int stateModel(double *pf, SIMDATA *pSim, double *pfstore, double fvap,
double lmVfgf, int nsteps, double minVRG) {
      double Q, Ve, Vfgf, dt;
      double kVRG, kMUS, kFAT;
      double nf[7], f[7];
      int i, n;
      // f is the state space vector
      // f(0): fraction of anesthetic in the circuit
      // f(1): fraction of anesthetic in the alveoli
      // f(2): fraction of anesthetic in the arterial compartment
      // f(3): fraction of anesthetic in the VRG
      // f(4): fraction of anesthetic in the muscle
      // f(5): fraction of anesthetic in the fat
      // f(6): fraction of anesthetic in the venous compartment
      // Q, Ve and lmVfgf are specified in L/min.
      // Convert L/min to L/sec
      Q = pSim->Q/60; Ve = pSim->Ve/60; Vfgf = lmVfgf/60;
      dt = pSim->dt;
      // Calculate the tissue time constants.
      kVRG = (pSim->lambda_vrg/pSim->lambda_blood) * (pSim->vol_vrg /
(Q*pSim->b_vrg));
      kMUS = (pSim->lambda_mus/pSim->lambda_blood) * (pSim->vol_mus /
(Q*pSim->b_mus));
      kFAT = (pSim->lambda_fat/pSim->lambda_blood) * (pSim->vol_fat /
(Q*pSim->b_fat));
      // Copy the state space data to the local variables.
      for (i=0 ; i<=6 ; i++) f[i]=pf[i];
      n = 0;
      while ((n < nsteps ) && ( f[3] > minVRG )) {
            n++;
            nf[0] = f[0] + dt*( (Vfgf*(fvap − f[0]) + Ve*(f[1]− f[0])) /
pSim->vol_ckt);
            nf[1] = f[1] + dt*( (Ve*(f[0] − f[1]) + Q*pSim-
>lambda_blood*(f[6] − f[2])) / pSim->vol_alv);
            nf[2] = nf[1];
            nf[3] = f[3] + dt*( (f[2] − f[3])/kVRG );
            nf[4] = f[4] + dt*( (f[2] − f[4])/kMUS );
            nf[5] = f[5] + dt*( (f[2] − f[5])/kFAT );
            nf[6] = (pSim->b_vrg*f[3]) + (pSim->b_mus*f[4]) + (pSim-
>b_fat*f[5]);
            for (i=0 ; i<=6 ; i++) f[i] = nf[i];
            if (pfstore != NULL) {
                  for (i=0 ; i<=6 ; i++) *(pfstore++)=nf[i];
            }
      }
      return n;
}
```

As can be appreciated from the code, the state model assumes that the concentrations of gases in the compartments follow first order kinetics, and uses first order kinetics and mass balance considerations to calculate the fraction of anesthetic gas in each physiological compartment. First, the cardiac output and minute ventilation are unit-converted so that they are expressed in terms of liters per second, instead of liters per minute. Then, a series of tissue time constants are calculated for the VRG, muscle, and fat based on the solubilities of the anesthetic agent in each compartment, the partition coefficients, and the volumes of the respective compartments. Once those basic calculations are done, the state model uses a while loop to iteratively calculate and update the anesthetic fractions in the compartments. The while loop has two conditions attached to it, such that the state model continues to calculate the compartmental anesthetic fractions while the VRG anesthetic concentration (f[3] in the above code) is greater than a minimum VRG concentration passed to the state model when it is called, and the number of times that the loop has been executed is less than a variable nsteps, which is also passed to the state model when it is called. Thus, the extent of the state model's calculations can be controlled either by specifying a VRG anesthetic concentration value to meet or by explicitly specifying the number of times that the state model loop is to be executed. The latter mode of control, specifying the number of times that the loop is to be executed, can be used to calculate the amount of time it will take a patient to reach a VRG concentration equal to ⅓ of the anesthetic agent's MAC value, as will be described below in more detail.

To make a single UDF, the makeSingleUDF function calls the state model function once with parameters that indicate time zero, a unit vaporizer setting (e.g., 1% vaporizer setting), and a time duration equal to the unit time duration. Then the state model is called again with parameters indicating a vaporizer setting of zero at a time of “unit duration+1.” Finally, the makeSingleUDF function searches the data for time at which the UDF peaks and returns that value. One implementation of this is as follows:

```
int makeSingleUDF(double *pudf, int udfLength, SIMDATA *pSim,
double Vfgf)
{
    int i, tpeak;
    double currentmax;
    double *pfstore, *pfi;
    // First, allocate memory for the arrays used to store the UDF
    pfstore = (double *)malloc( 7*(udfLength+1)*sizeof(double) );
    if (pfstore != NULL) {
        // Zero the initial state of the pfstore
        for (i=0; i<=6; i++) pfstore[i]=0;
        //Make the state model calls, first 1% at time zero, then
        //0% at unit time, store the data in pfstore.
    stateModel(pfstore,pSim,pfstore+7,1.0,Vfgf,pSim->unitDuration);
    stateModel(pfstore+7*pSim->unitDuration, pSim,pfstore+7*(pSim-
>unitDuration+1),0,pSim->minVfgf,udfLength-pSim->unitDuration);
        // Find the maximum and copy the VRG data to the UDF store
        currentmax = 0; tpeak = 0; pfi = pfstore + 10;
        for (i=1; i<=udfLength; i++) {
          if (pudf != NULL) *(pudf++) = *pfi;
          if ((*pfi)>currentmax) {
              currentmax = *pfi;
              tpeak = i;
          }
          pfi += 7;
        }
        free(pfstore);
        return tpeak;
    }
    else return −1; // Return negative tpeak if malloc failed.
}
```

Either once tasks 164 and 166 are complete, or if it is determined that the fresh gas flows and UDFs do not need to be recalculated (task 162:NO), method 150 continues with task 168, in which it is determined whether manual control over fresh gas flow and vaporizer settings is desired, or whether an automatic, target-based control mode is desired.

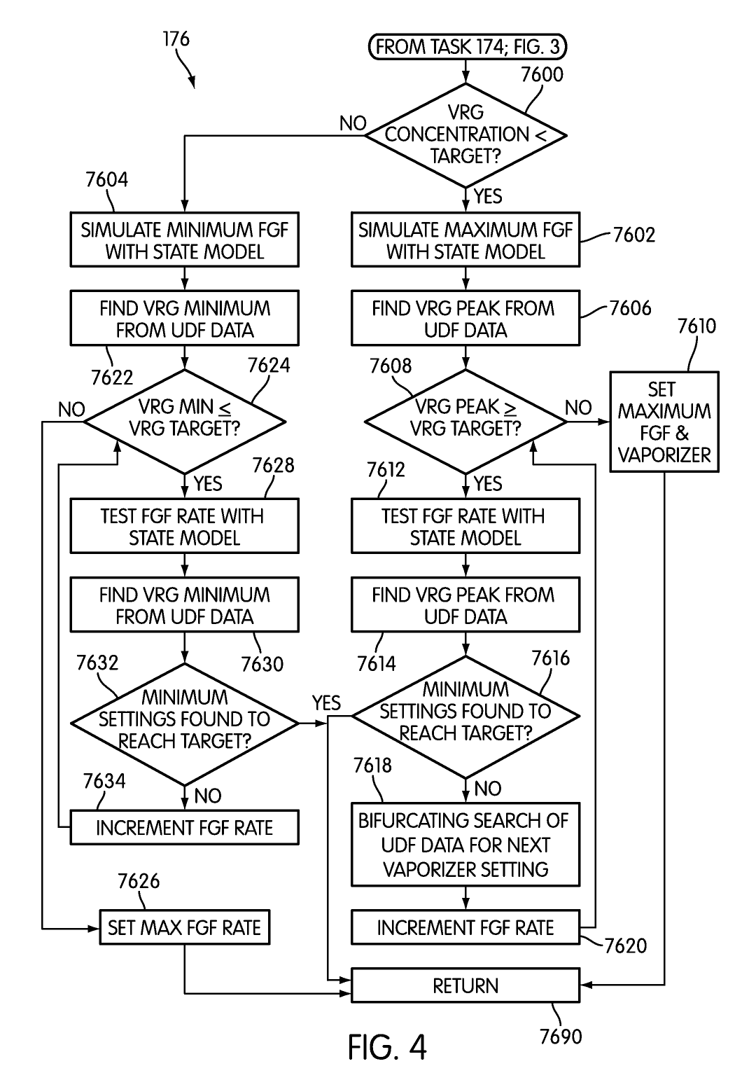

If automatic, target-based control mode is selected (task 168:AUTOMATIC), method 150 continues with tasks 170-174, which are essentially identical with tasks 162-166 described above, and checks to see whether settings have changed and the fresh gas flow rates and UDFs thus need to be recalculated. Following task 174, or if the settings have not changed (task 170:NO), method 150 continues with task 176, the main VRG target-based control algorithm. The VRG target-based control algorithm of task 176 has a number of sub-tasks, and those sub-tasks are illustrated in FIG. 4.

The VRG target-based control algorithm of task 176 begins with task 7600, a decision task. In task 7600, it is determined whether or not the VRG concentration is less than the target VRG concentration. If the VRG concentration is less than the target (task 7600:YES), the algorithm continues with task 7602; if the VRG concentration is not less than the target (i.e., it is greater than the target), the algorithm continues with task 7604.

Once it is determined that the VRG concentration is less than the target and, thus, anesthetic agent needs to be added to the circuit, the algorithm begins searching for the vaporizer settings and minimum fresh gas flow rate that will allow the VRG concentration to reach the target. This may be done by searching in any number of ways, but one of the most efficient ways to search for the desired parameters is by first checking a number of “edge cases.” For example, the controller 12 may first check the maximum FGF rate and vaporizer setting to see if it is possible to reach the target in the present iteration of task 176; if so, a more specific search may be executed to look for the minimum FGF and vaporizer settings that will reach the target.

Specifically, in task 7602 the state model is executed to simulate the effect of maximum fresh gas flow rate on the VRG concentration. Following task 7602, in task 7606, the VRG concentration peak with maximum fresh gas flow is determined by referencing the UDF data for the maximum flow rate, and the algorithm continues with task 7608, a decision task. In task 7608, if the VRG concentration peak from the UDF data is greater than or equal to the VRG target concentration (task 7608:YES), it is an indication that it is possible to reach the target VRG concentration in the present increment of time. (In the illustrated embodiment of the invention, the decisions made in task 176 are decisions for a single, discrete increment of time; the algorithm may be executed, for example, once a second.) If the VRG peak concentration is not greater than or equal to the VRG target concentration (task 7608:NO), then it is not possible to reach the VRG target concentration in the present increment of time, and control passes to task 7610, in which the algorithm simply sets the maximum fresh gas flow rate and vaporizer setting before returning to the other tasks of method 150 via task 7690.

As was noted above, once it has been established in task 7608 that it is possible to reach the VRG target concentration in the present increment of time, the algorithm begins iteratively testing flow rates from the array of possible flow rates, beginning with the minimum fresh gas flow rate, until the minimum FGF rate that will suffice to reach the VRG target concentration is found. In task 7612, the algorithm executes the state model with the present FGF rate that is being tested, and then in task 7614, the VRG peak concentration for that flow rate is determined from the existing UDF data. The algorithm continues with task 7616, a decision task. In task 7616, if the minimum fresh gas flow and vaporizer settings have been found (task 7616:YES), those settings are implemented before returning to the other tasks of method 150 via task 7690.

If the minimum fresh gas flow and vaporizer settings have not been found (task 7616:NO), the algorithm continues with task 7618 and executes a search of the UDF data for the next vaporizer setting to try. Any search algorithm may be used, although a bifurcating search has been found to be advantageous in at least some embodiments of the invention. One implementation of a bifurcating search suitable in at least some embodiments of the invention is as follows:

```
double bsm(double nmax, double tgt, double *x, double *y, int udfLength,
double tol) {
      double lo, hi, n, cn, cnmax;
      int i;
      lo = 0; hi = nmax; cnmax=0;
      for(;;) {
            n = 0.5*(lo+hi);
            for (i=0 ; i<udfLength ; i++) {
                  cn = x[i] + n*y[i];
                  if (cn>cnmax) cnmax = cn;
            }
            if (tgt>cnmax) lo = n;
            else if (tgt<cnmax) hi = n;
        else break;
            if ((hi-lo)<tol) break;
      }
      return n;
}
```

When the bifurcating search is executed, it takes the maximum vaporizer setting as the variable nmax, the VRG target concentration as the variable tgt, the time response in the VRG if no additional agent is administered as the variable x, the UDF as the variable y, the UDF length as the variable udfLength, and a search tolerance as the variable tol. The UDF is superposed on the time response in the VRG if no additional agent is added in a bifurcating search of the scaling parameter to calculate the appropriate vaporizer setting. The search ends if the difference between the high and low values is less than the specified tolerance.

Following that, the FGF rate is incremented in task 7620 before the algorithm returns to task 7608 and executes the loop again.

As was described above, if in task 7600, the VRG concentration was not less than the target, indicating that there is too much anesthetic agent in the circuit, no anesthetic agent needs to be added, and the algorithm attempts to find the minimum fresh gas flow rate that will bring the VRG concentration down to the target, as with task 7602, this process begins by checking “edge cases,” which, in this circumstance, involves first testing the effect of the minimum fresh gas flow rate. Specifically, in task 7604, the state model is executed to simulate the effect of the minimum fresh gas flow rate. Following that, in task 7622, the VRG minimum concentration is found by using the UDF data for the minimum flow rate.

The algorithm then continues with a decision task, in task 7624. In task 7624, if the VRG minimum concentration found using the UDF data is less than the VRG target concentration (task 7624:YES), it is an indication that the VRG target concentration can be reached in the present iteration. If the VRG minimum concentration found using the UDF data is not less than the VRG target concentration (task 7624:NO), then the VRG target concentration cannot be reached in the present iteration, and the algorithm sets the maximum fresh gas flow rate in task 7626 before returning to the other tasks of method 150 via task 7690.

After task 7624:YES, the algorithm continues with an iterative loop. Beginning with the minimum fresh gas flow rate, the algorithm executes the state model to test the effect of that flow rate, and finds the VRG minimum associated with that flow rate using the UDF data. If the minimum settings have been found (task 7632:YES), the algorithm implements those settings and returns to the other tasks of method 150 via task 7690. If the minimum settings have not been found (task 7632:NO), the algorithm increments the FGF rate in task 7634 and returns to task 7624 to continue the loop.

After the algorithm of task 176 is complete, method 150 continues in task 180 by running the state model with the FGF rate and vaporizer settings determined in task 176 in order to update the anesthetic concentrations in the other physiological compartments. Following that, in task 182, the method may update or store the history of the compartmental kinetics and settings. In task 184, the method updates the cost of the agent that has been expended, and also estimates the time it will take for the patient to awaken by calling the state model with the maximum fresh gas flow rate, a large number of time steps, and a VRG target value that is ⅓ of the MAC value for the anesthetic agent that is being used. Following task 184, the GUI is updated in task 186.

If after task 186, there has been no signal to terminate (task 188:NO), method 150 returns to task 160, the case timer is updated, and the method continues from that point. If there has been a signal to terminate and return (task 188:YES), method 150 does so via task 190.

In addition to the basic tasks described and illustrated in FIGS. 3 and 4, methods according to embodiments of the invention may include any number of other tasks, depending on the particular implementation and the desired functions of the controller 12. For example, as those of ordinary skill in the art will realize, method 150, as described above, “forward calculates” and determines gas administration characteristics using only the state model. Although the patient's mass is input and used by the state model, in the illustrated embodiment, the controller 12 operates without reference to any other physiological measurements or vital signs taken from the patient during the process. In many embodiments, if the state model that is used is sufficiently accurate, that approach may be sufficient to achieve the desired results. However, in other embodiments of the invention, the end-tidal anesthetic concentration, oxygen saturation levels, and other vital signs and physiological measurements may be taken and used by the controller 12 to improve the ability of the controller 12 to reach and maintain the desired targets. For example, cardiac output may be obtained directly from certain arterial pressure line sensors.

In some embodiments, the controller 12 may modify the state model depending on the condition or attributes of the patient. Any element of the state model may be modified or varied. For example, the compartment sizes used by the state model may be modified, such that patients with certain lung diseases would be assumed to have a different lung compartment size, morbidly obese patients would be assumed to have a larger fat compartment, and particularly muscular patients would be assumed to have a larger muscle compartment. In the lung compartment, ventilatory dead space may be determined for a particular patient and taken into account when determining lung compartment size.

The controller may also monitor inspired and expired gas concentrations to detect loss of circuit contents due to unexpected leakage from the circuit inside the anesthesia machine 10, leakage at the mask in mask ventilation, and deliberate or unanticipated disconnection of the patient 14 from the circuit 16. If leakage or another problem is detected, the controller may raise an alarm to warn the responsible personnel of the situation. In particular situations, if a hazard exists, the controller may be programmed to terminate the delivery of gases.

In the above description, the unit time interval was one second. When the controller 12 is being used in a simulation or an advisory mode, and the FGF and vaporizer settings are under the manual control of the anesthesiologist, the controller 12 may be programmed to use a longer unit time duration, allowing it to anticipate changes to settings over the next several minutes, so that there is sufficient time for the controller 12 to alert the anesthesiologist to make a change in the settings, and for the anesthesiologist to make that change.

While the invention has been described with respect to certain embodiments, the description is intended to be exemplary, rather than limiting. Modifications and changes may be made within the scope of the invention, which is defined by the appended claims.

1-28. (canceled)

29. A method for controlling the administration of an inhaled anesthetic agent, comprising:
a) determining gas administration characteristics, including at least a gas administration characteristic for the inhaled anesthetic agent, that are likely to result in a desired physiological anesthetic concentration or effect by simulating the uptake and behavior of the inhaled anesthetic agent using a physiological simulation; and
b) controlling the administration of one or more gases, including at least the inhaled anesthetic agent, to a patient in accordance with the gas administration characteristics obtained using the physiological simulation.

30. The method of claim 29, wherein the gas administration characteristics comprise a fresh gas flow rate and an anesthetic concentration or partial pressure.

31. The method of claim 30, wherein determining the gas administration characteristics comprises determining the minimum fresh gas flow rate and anesthetic concentration or partial pressure that are likely to result in a desired physiological concentration or effect.

32. The method of claim 31, wherein determining the gas administration characteristics comprises:
a) linearizing the effects of at least one of fresh gas flow rate or anesthetic concentration or partial pressure; and
b) searching for the minimum fresh gas flow rate and anesthetic concentration or partial pressure that are likely to result in a desired physiological concentration or effect.

33. The method of claim 32, wherein the linearizing comprises:
a) treating the fresh gas flow rate as a discrete variable with a defined set of possible values; and
b) for each one of the defined set of possible values, precalculating a unit distribution function.

34. The method of claim 33, wherein a unit distribution function is a function that describes the effect of a defined unit of anesthetic agent delivered for a defined unit of time at one of the possible values of the fresh gas flow rate.

35. The method of claim 30, wherein controlling the administration of one or more gases comprises controlling the fresh gas flow rate and the anesthetic concentration or partial pressure.

36. An inhaled anesthetic controller, comprising:
a) an interface adapted to allow the selection of a desired physiological concentration of an inhaled anesthetic agent or a desired physiological effect of the inhaled anesthetic agent;
b) a physiological simulator adapted (1) to accept the desired physiological concentration or desired physiological effect, and (2) to determine one or more gas administration characteristics based on a physiological model of the uptake and behavior of the inhaled anesthetic agent to meet the desired physiological concentration or desired physiological effect; and
c) an effector unit adapted to provide control signals to control the administration of one or more gases, including at least the inhaled anesthetic agent, in accordance with the determined gas administration characteristics.

37. The inhaled anesthetic controller of claim 36, wherein the one or more gas administration characteristics comprise a fresh gas flow rate and an anesthetic concentration or partial pressure.

38. The inhaled anesthetic controller of claim 37, wherein the physiological simulator is adapted to determine a minimum fresh gas flow rate and a minimum anesthetic concentration or partial pressure to meet the desired physiological concentration or the desired physiological effect.

39. The inhaled anesthetic controller of claim 36, wherein the physiological simulator is adapted to determine the minimal gas administration characteristics to meet the desired physiological concentration or desired physiological effect.

40. The inhaled anesthetic controller of claim 36, wherein the interface is adapted to allow manual control over the one or more gas administration characteristics.

41. The inhaled anesthetic controller of claim 36, wherein the controller is adapted to receive input from one or more sensors to monitor the administration of the one or more gases.

42. The inhaled anesthetic controller of claim 41, wherein the controller is adapted to detect one or more of a leak in an anesthesia circuit, a leak in an anesthesia machine, or a disconnection of the patient from the anesthesia circuit.

43. An anesthesia delivery system, comprising:
a) an anesthesia circuit adapted to deliver a mixture of gases, including an inhaled anesthetic agent, to a patient and to recover expired gases from the patient;
b) at least one sensor adapted to measure one or more characteristics of the mixture of gases; and
c) an anesthetic controller, including:
i) an interface adapted to allow the selection of a desired physiological concentration of the inhaled anesthetic agent or a desired physiological effect of the inhaled anesthetic agent,
ii) a physiological simulator adapted (1) to accept the desired physiological concentration or desired physiological effect, and (2) to determine one or more gas administration characteristics based on a physiological model of the uptake and behavior of the inhaled anesthetic agent, and
iii) an effector unit adapted to provide control signals to the anesthesia circuit to control the administration of the mixture of gases in accordance with the determined gas administration characteristics to meet the desired physiological concentration or desired physiological effect.

44. The anesthesia delivery system of claim 43, wherein the one or more gas administration characteristics determined by said physiological simulator comprise a fresh gas flow rate and an anesthetic concentration or partial pressure.

45. The anesthesia delivery system of claim 44, wherein said physiological simulator is adapted to determine a minimum fresh gas flow rate and a minimum anesthetic concentration or partial pressure to meet the desired physiological concentration or the desired physiological effect.

46. The anesthesia delivery system of claim 43, wherein said physiological simulator is adapted to determine the minimal gas administration characteristics to meet the desired physiological concentration or desired physiological effect.

47. The anesthesia delivery system of claim 43 wherein said interface is adapted to allow manual control over the one or more gas administration characteristics.

48. The anesthesia delivery system of claim 43, wherein said anesthetic controller is adapted to receive input from one or more sensors to monitor the administration of the one or more gases.
